(19) United States
(12) Patent Application Publication
(10) Pub. No.: US 2025/0064076 A1
(43) Pub. Date: Feb. 27, 2025

(54) FERMENTED CAMOMILE BEVERAGE

(71) Applicant: SOCIETE DES PRODUITS NESTLE S.A., Vevey (CH)

(72) Inventors: JIYUAN ZHANG, Lausanne (CH); CYRIL MOCCAND, Lully (CH); AXEL ANDRE, Chexbres (CH)

(21) Appl. No.: 18/721,401

(22) PCT Filed: Dec. 12, 2022
(86) PCT No.: PCT/EP2022/085341

§ 371 Date: Jun. 18, 2024

(30) Foreign Application Priority Data
Dec. 21, 2021 (EP) 21216396.8
Jan. 14, 2022 (EP) 22151532.3

(51) Int. Cl. A23F 3/16; A23F 3/34

(52) U.S. Cl. CPC A23F 3/166; A23F 3/34

(57) ABSTRACT

The present invention relates to a beverage composition comprising fermented camomile or fermented camomile extract. A further aspect of the invention is a method of preparing a beverage composition.

### FIELD OF THE INVENTION

The present invention relates to a beverage composition comprising fermented camomile or fermented camomile extract. A further aspect of the invention is a method of preparing a beverage composition.

### BACKGROUND OF THE INVENTION

Camomile tea is a herbal tea made from dried camomile flowers and hot water. It has long been drunk for its taste and as a traditional folk remedy for a wide range of health issues such as gastrointestinal problems. In ancient Egypt, camomile tea was prescribed as a cold remedy.

Camomile tea has an earthy flavour which is not always appreciated by consumers. Many people desire new flavours in their beverages and sweet tastes, but would prefer not to consume beverages with added flavouring, especially synthetic flavourings or sugary fruit juices. The provision of camomile beverages with a less strong earthy note would therefore be of commercial value.

Any reference to prior art documents in this specification is not to be considered an admission that such prior art is widely known or forms part of the common general knowledge in the field. As used in this specification, the words “comprises”, “comprising”, and similar words, are not to be interpreted in an exclusive or exhaustive sense. In other words, they are intended to mean “including, but not limited to”.

### SUMMARY OF THE INVENTION

An object of the present invention is to improve the state of the art and to provide a new product to overcome at least some of the inconveniences described above, or at least to provide a useful alternative. The object of the present invention is achieved by the subject matter of the independent claims. The dependent claims further develop the idea of the present invention.

Accordingly, the present invention provides in a first aspect a beverage composition comprising fermented camomile or fermented camomile extract wherein the weight ratio of the sum of 3-methylbutyl acetate and 3-methylbutanol to 3-methylbutanal is greater than 2000.

A second aspect of the invention provides a container for use in a beverage preparation device, the container containing the beverage composition of the invention.

In a third aspect, the invention provides a method of preparing a beverage composition comprising;
a. steeping camomile in water to form an aqueous camomile extract and steeped camomile; and
b. fermenting the aqueous camomile extract in the presence or absence of the steeped camomile;
   wherein the fermentation is performed by yeast comprising Pichia yeast.

A further aspect of the invention provides use of camomile fermented with Pichia kluyveri NYSC 5485 (CNCM I-5525) to produce a beverage.

Camomile flowers can be made into a beverage infusion by steeping the dried flowers in hot water. These beverages have earthy notes with subtle sweetness. The inventors were surprised to find that, through fermentation with Pichia yeast, the camomile beverage remained low in ethanol, but the flavours generated by fermentation covered the earthy notes and enhanced the sweetness, without introducing off-notes. In particular, the “green” and “woody” note that some consumers don't appreciate in camomile beverages was reduced.

### DETAILED DESCRIPTION OF THE INVENTION

Consequently the present invention relates in part to a beverage composition comprising (for example consisting of) fermented camomile or fermented camomile extract wherein the weight ratio of the sum of 3-methylbutyl acetate and 3-methylbutanol to 3-methylbutanal is greater than 2000, for example greater than 4000, for example greater than 8000, for example greater than 10000, for example greater than 12000, for example greater than 14000, for example greater than 16000, for example greater than 18000, for example greater than 20000, for further example greater than 22000. The aldehyde 3-methylbutanal contributes a malty, solvent-like note.

The ratio can be calculated from the weight concentrations in parts per million as follows:

$$\frac{[ 3 - methylbutyl acetate ] + [ 3 - methylbutanol ]}{[ 3 - methylbutanal ]}$$

Higher esters such as 3-methylbutyl acetate are initially produced during the fermentation of sugars with Pichia, but as the fermentation proceeds, higher alcohols such as 3-methylbutanol are produced. A high ratio of the sum of 3-methylbutyl acetate and 3-methylbutanol to 3-methylbutanal corresponds to a pleasant aroma balance with reduced malty, beer and solvent notes in the fermented beverage, masking the earthy note. The “tea” note that some consumers do not appreciate is also masked by higher ratios of the sum of 3-methylbutyl acetate and 3-methylbutanol to 3-methylbutanal.

In the context of the present invention the term camomile refers to the flowers of the daisy-like plant of the family Asteraceae used to make the herbal tea “camomile tea”. The camomile may be Matricaria recutita (German camomile) or Anthemis nobilis (English camomile). Preferably the camomile is Matricaria recutita. The word camomile is sometimes spelt as chamomile. Camomile contains polyphenols such as apigenin, quercetin, patuletin, and luteolin.

In the context of the present invention the term “fermentation” refers to a process in which the activity of microorganisms brings about a change (typically a desirable change) to a foodstuff or beverage. The fermentation may be with yeasts and/or bacteria. The fermentation may be anaerobic or aerobic. Fermentation is one of the oldest means of preserving and enhancing foods.

The term “fermented camomile” refers to camomile which has been subjected to a process in which the activity of microorganisms such as yeasts brings about a chemical change in the organic components of the camomile, typically a desirable change. The fermented camomile may have been fermented in an aqueous medium, for example with yeast. The fermented camomile may be a dried camomile which had been added to an aqueous fermentation medium and fermented before being separated from the aqueous fermentation medium and dried. The fermented camomile may have been fermented in a “solid-state” fermentation, for example adding a starter culture of yeast to camomile, for example to fresh camomile flowers.

The term “camomile extract” refers to material extracted from camomile, for example an aqueous extract of camomile. The term “fermented camomile extract” refers to an extract of camomile where the camomile extract has been fermented, for example an aqueous camomile extract where the aqueous extract has been fermented, such as by yeast. The fermented camomile extract may be a dried fermented camomile extract.

In an embodiment the beverage composition (for example the beverage) has a weight ratio of the sum of 2-methylbutyl acetate and 2-methylbutanol to 2-methylbutanal is greater than 2000, for example greater than 4000, for example greater than 6000, for example greater than 7000, for further example greater than 8000.

The ratio can be calculated from the weight concentrations in parts per million as follows:

$$\frac{[ 2 - methylbutyl acetate ] + [ 2 - methylbutanol ]}{[ 2 - methylbutanal ]}$$

In an embodiment the beverage composition (for example the beverage) has a weight ratio of the sum of isobutyl acetate and isobutanol to isobutanal greater than 400, for example greater than 500, for example greater than 600, for example greater than 700, for example greater than 800, for example greater than 900, for further example greater than 950. Isobutyl acetate (IUPAC name 2-methylpropyl ethanoate) is the acetate ester of isobutanol. The alcohol isobutanol has the IUPAC name 2-methyl-propan-1-ol. The aldehyde isobutanal is sometimes known as 2-methylpropanal and contributes a malty note.

The ratio can be calculated from the weight concentrations in parts per million as follows:

$$\frac{[ isobutyl acetate ] + [ isobutanol ]}{[ isobutanal ]}$$

Higher esters such as isobutyl acetate are initially produced during the fermentation of sugars with Pichia, but as the fermentation proceeds, higher alcohols such as isobutanol are produced. A high ratio of the sum of isobutyl acetate and isobutanol to isobutanal corresponds to a pleasant aroma balance and less malty beer note in the fermented beverage.

In an embodiment the beverage composition (for example the beverage) has a weight ratio of the sum of 3-methylbutyl acetate, 2-methylbutyl acetate, isobutyl acetate, 2-phenylethyl acetate and 2-phenylethanol to 2,3-butanedione greater than 60, for example greater than 200, for example greater than 400, for example greater than 500, for example greater than 600, for example greater than 700, further example greater than 800. This ratio can be calculated from the weight concentrations in parts per million as follows:

$$\frac{\begin{matrix}[ 3 - methylbutyl acetate ] + [ 2 - methylbutyl acetate ] + \\ \begin{matrix}[ isobutyl acetate ] + [ 2 - phenylethyl acetate ] + \\ [ 2 - phenylethanol ]\end{matrix}\end{matrix}}{[ 2 , 3 - butanedione ]}$$

The compound 2,3-butanedione is also known as diacetyl. It provides a buttery or fatty note. A high ratio of the sum of 3-methylbutyl acetate, 2-methylbutyl acetate, isobutyl acetate, 2-phenylethyl acetate and 2-phenylethanol to 2,3-butanedione corresponds to enhanced fruity and floral notes without the buttery and fatty notes that can be perceived as off flavour in a fermentation.

In an embodiment, the beverage composition comprises less than 10 g/kg acetic acid on a dry basis, for example less than 1 g/kg acetic acid on a dry basis, for further example less than 0.5 g/kg acetic acid on a dry basis. The acid acid “bite” in the flavour profile of beverages such as kombucha or kefir is not always desired by consumers who seek a gentler flavour profile.

Many consumers seek beverages with a complex, refreshing taste as a replacement to alcoholic drinks. In an embodiment, the beverage composition has an ethanol content below 1.2 wt %, for example below 0.5 wt %, for example below 0.2 wt %, for further example below 0.05 wt %.

The beverage composition may be selected from the group consisting of a ready to drink beverage, a beverage liquid concentrate, a soluble beverage powder, dried aromatic plant material and combinations of these.

The ready to drink beverage may be a fermented camomile infusion or a water flavoured with fermented camomile. The ready to drink beverage may additionally comprise other components such as flavourings or stabilizers. The ready to drink beverage may be carbonated. The ready to drink beverage may additionally comprise fruit or fruit juice, such as strawberry, orange, lemon, lime, peach, pomegranate, watermelon, blackberry or cranberry. The ready to drink beverage may additionally comprise herbs such as mint, verbena or basil. The ready to drink beverage may additionally comprise flower extracts such as hibiscus or rose. The ready to drink beverage may comprise added vitamins and minerals. The ready to drink beverage may comprise beneficial microorganisms, replicating or non-replicating. The beneficial microorganisms may for example be probiotics.

Beverage preparation devices (for example beverage preparation machines) which accommodate extractable portioned ingredients provide a convenient method of preparing beverages. Such portioned ingredients are generally packed in a container, configured for example as a pod, pad, sachet, pouch, capsule or the like. An aspect of the invention provides a container for use in a beverage preparation device, the container containing the beverage composition of the invention. The container being for the preparation of a beverage when inserted into a beverage preparation device. The container may for example be a beverage capsule, among other configurations. In an embodiment, the container contains the beverage composition of the invention. For example the container may contain dried fermented camomile or dried fermented camomile extract. The container may contain dried fermented camomile and/or dried fermented camomile extract, these may be combined with other dry ingredients such as tea, herbal tea, dried fruit, soluble coffee or roast and ground coffee.

An aspect of the invention provides a method of preparing a beverage composition comprising;
a. steeping camomile in water to form an aqueous camomile extract and steeped camomile; and
b. fermenting the aqueous camomile extract in the presence or absence of the steeped camomile;
   wherein the fermentation is performed by yeast comprising Pichia yeast.

The yeast may be predominantly Pichia yeast, for example, more than 50% of the microorganism colony forming units present during fermentation may be Pichia. For example more than 50% of the yeast colony forming units may be Pichia, for example more than 60, 70, 80, 90% of the yeast colony forming units may be Pichia. For example, essentially all the yeast colony forming units present during fermentation may be Pichia, for further example, essentially all the microorganism colony forming units present during fermentation may be Pichia.

The beverage composition prepared by the method of the invention may be a camomile beverage, for example a beverage comprising fermented camomile or fermented camomile extract. The beverage composition prepared by the method of the invention may have an ethanol content below 1.2 wt %, for example below 0.5 wt %, for example below 0.2 wt %, for further example below 0.05 wt %. In an embodiment, the beverage composition prepared by the method of the invention is the beverage composition of the invention.

The term steeping refers to immersing and soaking a material in liquid. The camomile may be steeped in water for at least 2 minutes, for example at least 3 minutes, for example at least 4 minutes, for example at least 5 minutes, for example at least 10 minutes, for example at least 15 minutes, for example at least 20 minutes, for example at least 30 minutes, for example at least 60 minutes, for further example at least 120 minutes. During the steeping process, components of the camomile are extracted into the water. The steeping may be performed at a temperature between 4° C. and 98° C., for example between 20° C. and 95° C., for example between 60° C. and 95° C. Although camomile contains some fermentable sugar, further fermentable sugar may be added to the aqueous camomile extract, for example sucrose or glucose. The fermentable sugar may be fructose or glucose. The fermentable sugar may be in the form of honey. For example fermentable sugar may be added to the aqueous camomile extract at a level of between 2 and 10 wt. %, for example between 3 and 7 wt. %. The camomile may be dried and/or ground before steeping.

When fermentation is to be performed in the absence of the steeped camomile, the camomile may for example be steeped in water using a continuous counter-current extraction system. A further option is to steep the camomile in water using a batch process, with the steeped camomile being separated from the aqueous camomile extract using a filter system. The ratio of camomile on a dry basis to water during steeping may be between 1:1 and 1:100 by weight, for example between 1:2 and 1:95 by weight, for example between 1:10 and 1:90 by weight, for example between 1:30 and 1:70 by weight, for further example between 1.3 and 1:6.

After steeping, the temperature of the aqueous camomile extract is adjusted to a temperature suitable for yeast growth, for example between 20° C. and 37° C., for example between 25° C. and 35° C., for example between 28° C. and 32° C. The temperature may for example be adjusted using a laminar flow heat exchanger. Yeast is added to the aqueous camomile extract to commence fermentation. Fermenting the aqueous camomile extract maybe performed at a temperature between 25° C. and 35° C., for example between 28° C. and 32° C. Fermenting the aqueous camomile extract may be performed for at least 2 hours, for example at least 6 hours, for example at least 12 hours, for example at least 24 hours, for further example at least 48 hours.

The fermentation may be performed at a pH between 3 and 8, for example between 4 and 6.

The fermentation may be performed in the presence of a nitrogen source such as yeast extract or yeast peptone. The fermentation may be performed in the presence of added amino acids such as valine, leucine, isoleucine and/or phenylalanine, for example at a level of between 0.05 wt. % and 0.1 wt. % of the aqueous camomile extract.

The fermentation may be performed in anaerobic, microaerobic or aerobic conditions. For example, the partial oxygen pressure in the fermenter may be from 0 to 30%, for example from 1 to 20%, for further example from 2 to 10%.

Preferably the camomile has not been subjected to chemical processing such as hydrolysis before fermentation.

The fermentation may be performed by adding one or more yeasts at an initial combined yeast level of at least 10³ CFU/g, for example at least 10⁴ CFU/g, for example at least 10⁵ CFU/g, for example at least 10⁶ CFU/g, for further example at least 10⁷ CFU/g.

The fermentation may be performed in the absence of acetic acid bacteria to avoid generating an acid “bite” in the flavour profile, such as may be obtained with kefir fermentations. In an embodiment the fermentation is performed in the absence of lactic acid bacteria.

In an embodiment the fermentation is performed in the absence of Saccharomyces subspecies. For example the fermentation may be performed in the absence of Saccharomyces cerevisiae. S. cerevisiae is used for winemaking and beer brewing. In addition to generating alcohol which may not be desired, S. cerevisiae generates a “fermented”, winey, yeasty flavour note rather than a fresh fruity note.

In an embodiment the fermentation is performed in the absence of a yeast selected from the group consisting of Zygosaccharomyces bailii, Schizosaccharomyces pombe, Torulaspora delbreuckii, Rhodotorula mucilaginosa, Brettanomyces bruxellensis and Candida stellate. These yeasts are commonly found in kombucha “tea-fungus” cultures.

The inventors found that fermentation of camomile with Pichia kluyveri gave particularly good results. A beverage composition comprising camomile or camomile extract fermented with Pichia kluyveri had a pleasant fruity flavour with less grassy note than the unfermented beverage and an enhanced sweetness perception.

Pichia kluyveri occurs naturally around the world, for example on olives, grapes and coffee. In an embodiment the Pichia yeast is predominantly Pichia kluyveri, for example, more than 50% of the microorganism colony forming units present during fermentation may be Pichia kluyveri. For example more than 50% of the yeast colony forming units may be Pichia kluyveri, for example more than 60, 70, 80, 90% of the yeast colony forming units may be Pichia kluyveri. For example, essentially all the yeast colony forming units present during fermentation may be Pichia kluyveri, for further example, essentially all the microorganism colony forming units present during fermentation may be Pichia kluyveri.

Pichia kluyveri NCYC 246 (alternatively designated CBS 188) is the type-strain of Pichia kluyveri. It is publicly available, for example from the National Collection of Yeast Cultures, Quadram Institute Bioscience, Norwich, UK.

In an embodiment the Pichia yeast is Pichia kluyveri NCYC 246. For example more than 50% of the yeast colony forming units present during fermentation may be Pichia kluyveri NCYC 246. For example, more than 50% of the microorganism colony forming units present during fermentation may be Pichia kluyveri NCYC 246. For example, essentially all the yeast colony forming units present during fermentation may be Pichia kluyveri NCYC 246. For further example, essentially all the microorganism colony forming units present during fermentation may be Pichia kluyveri NCYC 246.

Pichia kluyveri NCYC 2781 (alternatively designated Maize Silage Isolate 86) is publicly available, for example from the National Collection of Yeast Cultures, Quadram Institute Bioscience, Norwich, UK.

In an embodiment the Pichia yeast is Pichia kluyveri NCYC 2781. For example more than 50% of the yeast colony forming units present during fermentation may be Pichia kluyveri NCYC 2781. For example, more than 50% of the microorganism colony forming units present during fermentation may be Pichia kluyveri NCYC 2781. For example, essentially all the yeast colony forming units present during fermentation may be Pichia kluyveri NCYC 2781. For further example, essentially all the microorganism colony forming units present during fermentation may be Pichia kluyveri NCYC 2781.

Pichia kluyveri is widely distributed in nature, for example being found on fruits such as coffee cherries and olives. Pichia kluyveri NYSC 5485, collected and isolated from a coffee fermentation in Nicaragua, was deposited with the Collection Nationale de Cultures de Microorganismes (CNCM), Institut Pasteur, 25 rue du Docteur Roux, F-75724 PARIS Cedex 15, France, on 23 Jun. 2020 and given the deposit number CNCM I-5525.

In an embodiment, the yeast is Pichia kluyveri NYSC 5485 (CNCM I-5525). For example more than 50% of the yeast colony forming units present during fermentation may be Pichia kluyveri NYSC 5485 (CNCM I-5525). For example, more than 50% of the microorganism colony forming units present during fermentation may be Pichia kluyveri NYSC 5485 (CNCM I-5525). For example, essentially all the yeast colony forming units present during fermentation may be Pichia kluyveri NYSC 5485 (CNCM I-5525), For further example, essentially all the microorganism colony forming units present during fermentation may be Pichia kluyveri NYSC 5485 (CNCM I-5525).

The inventors have found that especially good results were obtained by fermenting camomile with Pichia kluyveri NYSC 5485. Fermenting camomile with Pichia kluyveri NYSC 5485 resulted in a higher weight ratio of the sum of 3-methylbutyl acetate and 3-methylbutanol to 3-methylbutanal, corresponding to an enhanced aroma balance.

The fermentation may be performed by adding Pichia kluyveri, for example Pichia kluyveri NYSC 5485 (CNCM I-5525), at an initial yeast level of at least 10³ CFU/g, for example at least 10⁴ CFU/g, for example at least 10⁵ CFU/g, for example at least 10⁶ CFU/g, for further example at least 10⁷ CFU/g.

The genetic sequence of Pichia kluyveri NYSC 5485 (CNCM I-5525) was analysed using PacBio sequencing technology. DNA purification was performed using a Qiagen Gentra Puregene Yeast kit. Twelve DNA fragments were identified. These may be individual chromosomes; 11 chromosomes and 1 mitochondrion DNA (SEQ ID NO:4). The genetic sequences are filed in electronic form as SEQ ID NO:1-SEQ ID NO:12.

In an embodiment of the method of the invention, the Pichia yeast comprises a genetic sequence having at least 98% (for example at least 99% identity) to SEQ ID NO:1, a genetic sequence having at least 98% identity (for example at least 99% identity) to SEQ ID NO:2, a genetic sequence having at least 98% identity (for example at least 99% identity) to SEQ ID NO:3, a genetic sequence having at least 98% identity (for example at least 99% identity) to SEQ ID NO:5, a genetic sequence having at least 98% identity (for example at least 99% identity) to SEQ ID NO:6, a genetic sequence having at least 99% identity to SEQ ID NO:7, a genetic sequence having at least 98% identity (for example at least 99% identity) to SEQ ID NO:8, a genetic sequence having at least 98% identity (for example at least 99% identity) to SEQ ID NO:9, a genetic sequence having at least 99% identity to SEQ ID NO:10, a genetic sequence having at least 98% identity (for example at least 99% identity) to SEQ ID NO:11, or a genetic sequence having at least 98% identity (for example at least 99% identity) to SEQ ID NO:12.

In an embodiment of the method of the invention, the Pichia yeast comprises a genetic sequence having at least 98% (for example at least 99% identity) to SEQ ID NO:1, a genetic sequence having at least 98% identity (for example at least 99% identity) to SEQ ID NO:2, a genetic sequence having at least 98% identity (for example at least 99% identity) to SEQ ID NO:3, a genetic sequence having at least 99.5% identity to SEQ ID NO:4, a genetic sequence having at least 98% identity (for example at least 99% identity) to SEQ ID NO:5, a genetic sequence having at least 98% identity (for example at least 99% identity) to SEQ ID NO:6, a genetic sequence having at least 99% identity to SEQ ID NO:7, a genetic sequence having at least 98% identity (for example at least 99% identity) to SEQ ID NO:8, a genetic sequence having at least 98% identity (for example at least 99% identity) to SEQ ID NO:9, a genetic sequence having at least 99% identity to SEQ ID NO:10, a genetic sequence having at least 98% identity (for example at least 99% identity) to SEQ ID NO:11 and a genetic sequence having at least 98% identity (for example at least 99% identity) to SEQ ID NO:12.

In an embodiment of the method of the invention, the Pichia yeast comprises a genetic sequence having a global nucleotide identity at least 98% (for example at least 99%) to the combination of SEQ ID NOs:1-12. By the term “combination of SEQ ID NOs:1-12” it is meant that the sequence identity comparison is performed against all the individual DNA fragments as if concatenated into a single sequence.

In an embodiment the Pichia yeast comprises a genetic sequence having at least 98% (for example at least 99%) identity to SEQ ID NO:1. In an embodiment the Pichia yeast comprises a genetic sequence having at least 98% (for example at least 99%) identity to SEQ ID NO:2. In an embodiment the Pichia yeast comprises a genetic sequence having at least 98% (for example at least 99%) identity to SEQ ID NO:3. In an embodiment the Pichia yeast comprises a genetic sequence having at least 98% (for example at least 99%) identity to SEQ ID NO:5. In an embodiment the Pichia yeast comprises a genetic sequence having at least 98% (for example at least 99%) identity to SEQ ID NO:6. In an embodiment the Pichia yeast comprises a genetic sequence having at least 99% identity to SEQ ID NO:7. In an embodiment the Pichia yeast comprises a genetic sequence having at least 98% (for example at least 99%) identity to SEQ ID NO:8. In an embodiment the Pichia yeast comprises a genetic sequence having at least 98% (for example at least 99%) identity to SEQ ID NO:9. In an embodiment the Pichia yeast comprises a genetic sequence having at least 99% identity to SEQ ID NO:10. In an embodiment the Pichia yeast comprises a genetic sequence having at least 98% (for example at least 99%) identity to SEQ ID NO:11. In an embodiment the Pichia yeast comprises a genetic sequence having at least 98% (for example at least 99%) identity to SEQ ID NO:12.

The names by which yeasts are referred to may change with time. It should be understood that the yeast according to the method of the invention may comprise the genetic sequence identities described in the preceding paragraphs for the Pichia yeast of the invention regardless of the name applied to the yeast.

An aspect of the invention provides a method of preparing a beverage composition comprising; a) steeping camomile in water to form an aqueous camomile extract and steeped camomile; and b) fermenting the aqueous camomile extract in the presence or absence of the steeped camomile; wherein the fermentation is performed by a yeast comprising a genetic sequence having a global nucleotide identity at least 98% (for example at least 99%) to the combination of SEQ ID NOs:1-12.

In an embodiment, the aqueous camomile extract is separated from the steeped camomile after fermentation. For example the steeped camomile may be separated from the aqueous camomile extract using a filter system. After fermentation, the aqueous camomile extract becomes fermented camomile extract.

The fermented camomile extract may be packaged as a ready-to-drink beverage, for example packaged in a bottle or a can. The fermented camomile extract may have a concentration of between 2 and 60 wt. % total dissolved solids, for example a concentration of 2 to 25 wt. % total dissolved solids. Additional ingredients such as flavours and preservatives may be added to the fermented camomile extract before it is packaged as a ready-to-drink beverage. The fermented camomile extract may be heat-treated to de-activate or kill the yeast and any spoilage organisms before being packaged. The fermented camomile extract may be passed through a filter to remove the yeast and any spoilage organisms before being packaged.

In an embodiment the aqueous camomile extract is concentrated after being fermented, for example to be packaged as a liquid concentrate suitable to be diluted by the consumer before consumption. The aqueous camomile extract may be concentrated as an intermediate product for the manufacture of ready-to-drink products, for example in a different location. The fermented camomile extract may have a concentration of 25 to 60 wt. % total dissolved solids, for further example a concentration of 50 to 60 wt. % total dissolved solids. The dissolved solids may for example be measured by passing the extract through a fine filter to remove non-dissolved solids, weighing the filtrate to establish the total weight, evaporating the filtrate and then weighing the residue to obtain the weight of the dissolved solids.

The aqueous camomile extract after fermentation (for example the fermented camomile extract) may be concentrated to form a powder, such as a soluble beverage powder. For example, the aqueous camomile extract after fermentation (the fermented camomile extract) may be dried by spray drying or freeze drying to form a soluble beverage powder. For example, the aqueous camomile extract after fermentation may be spray dried together with a carrier such as maltodextrin to form a soluble beverage powder. The soluble beverage powder may be packaged and sold as such, or it may be used as an intermediate product for the manufacture of ready-to-drink products, for example in a different location. The soluble beverage powder may be filled into a container for use in a beverage preparation device. The soluble beverage powder may be combined with other ingredients, for example dry ingredients such as tea, herbal tea, dried fruit, soluble coffee or roast and ground coffee. For example, the soluble beverage powder may be combined with other ingredients and filled into a container for use in a beverage preparation device. The soluble beverage powder may be combined with milk powder or beverage creamer powder and optionally sugar to form a beverage mix. After fermenting the aqueous camomile extract in the presence of the steeped camomile, the steeped camomile becomes fermented camomile. In an embodiment, the steeped camomile, present during the fermentation of the aqueous camomile extract, is separated from the aqueous camomile extract after fermentation and then dried, for example using a rotary drier. The dried steeped camomile may be filled into a container for use in a beverage preparation device, or filled into a permeable bag such as a “tea-bag”. The dried steeped camomile may be combined with other dry ingredients such as tea, herbal tea, dried fruit, soluble coffee or roast and ground coffee. In the context of the present invention, the term tea refers to the dried leaves, leaf-buds, twigs or stems of the plant Camellia sinensis. The tea may for example be C. sinensis var. sinensis, or C. sinensis var. assamica. The term “herbal tea” refers to infusions made from the dried flowers, fruit, leaves, seeds or roots of plants other than Camellia sinensis.

In an embodiment, the steeped camomile, present during the fermentation of the aqueous camomile extract, is dried together with the aqueous camomile extract after fermentation, for example by freeze drying or spray drying. The dried steeped camomile (e.g. dried fermented camomile) together with dried aqueous camomile extract after fermentation (e.g. dried fermented camomile extract) may be filled into a container for use in a beverage preparation device, or filled into a permeable bag such as a “tea-bag”. The dried steeped camomile and dried aqueous camomile extract after fermentation may be combined with other dry ingredients such as tea, herbal tea, dried fruit, soluble coffee or roast and ground coffee.

An aspect of the invention provides for the use of camomile fermented with Pichia 15 kluyveri NYSC 5485 (CNCM I-5525) to produce a beverage.

Fermented camomile according to the invention may be obtained in a “solid-state” fermentation, for example adding a starter culture of Pichia yeast to camomile, for example fresh camomile flowers which have not been dried. The term “solid-state” fermentation refers to fermentation taking place on solid materials. There is liquid water present in a solid-state fermentation, but the term is used in contrast to a liquid fermentation such as may occur in aqueous solution in a fermentation vessel.

An aspect of the invention provides a method of preparing a beverage composition comprising;
adding a starter culture of yeast to camomile, wherein the starter culture provides at least 10⁴ CFU (for example at least 10⁵ CFU, for further example at least 10⁶ CFU) of yeast per gram of camomile;
fermenting the camomile;
drying the fermented camomile to form fermented camomile; and either;
extracting the fermented camomile with water to form a ready-to-drink beverage, or
filling the fermented camomile into a container, for example a container being for the preparation of a beverage when inserted into a beverage preparation device.

The starter culture of yeast may be added as a liquid or a powder to the camomile. The camomile flowers may have a moisture content of between 45 and 55 wt. % when the starter culture of yeast is added. The flowers may be treated with steam before fermentation to reduce the quantity of environmental microorganisms before adding yeast. The fermentation may occur at a solids content greater than 40 wt. %, for example greater than 45 wt. %, for further example greater than 50 wt. %.

Drying the fermented camomile may commence while some fermentation continues. For example, the camomile flowers may be placed in a container such as a tank, the starter culture added and then fermentation allowed to proceed for 1-4 days before the fermented camomile is removed from the container and dried. For further example, the starter culture may be added to the camomile flowers before the camomile flowers are slowly dried, for example on a rack or suspended tray. Once fermentation has started, the camomile flowers may be slowly dried whilst fermentation continues. For example the fermented camomile flowers may be dried to a moisture content of below 20 wt. % (for example below 15 wt. %) over a period of between 1 and 30 days, for example between 4 and 20 days. The drying temperature may be less than about 40° C., for example between about 25° C. and 35° C. A fan can be used to blow dry air over the material until the camomile flowers are dry. The fermented camomile flowers may be dried in the sun.

The yeast may be Pichia, for example Pichia kluyveri, for further example Pichia kluyveri NYSC 5485.

The container for the preparation of a beverage when inserted into a beverage preparation device may be in the form of a pod, pad, sachet, pouch, capsule or the like.

The ready-to-drink beverage may be filled into a bottle or can. The fermented camomile may be combined with other extractable materials such as tea, herbal tea, dried fruit, soluble coffee or roast and ground coffee before being extracted with water to form a ready-to-drink beverage.

The fermented camomile may be combined with tea, herbal tea, dried fruit, soluble coffee or roast and ground coffee in a container for the preparation of a beverage when inserted into a beverage preparation device.

Those skilled in the art will understand that they can freely combine all features of the present invention disclosed herein. In particular, features described for the product of the present invention may be combined with the method of the present invention and vice versa. Further, features described for different embodiments of the present invention may be combined. Where known equivalents exist to specific features, such equivalents are incorporated as if specifically referred to in this specification.

Further advantages and features of the present invention are apparent from the figures and non-limiting examples.

### EXAMPLES

### Example 1: Method of Fermenting Camomile

An infusion was prepared using organic camomile (Chamomilla recutita) (Coop Naturaplan, Switzerland) as starting material. A hot infusion was firstly made by adding 20 g of dry starting material in 1000 ml of water at 100° C., and steeping for 10 min. Afterwards, the infusion was placed in an ice bath until the temperature fell below 30° C. The infusion was then filtered through sieves to remove additional pieces. The filtrate (aqueous camomile extract) was distributed equally as control (Ref) or for different fermentation variants.

Fermented camomile was made by fermenting the infusion with glucose (2% m/m) and different starter cultures, including Pichia kluyveri NYSC 5485 (CNCM 1-5525), P. kluyveri NCYC 246 (the type strain of Pichia kluyveri), P. kluyveri NCYC 2781, commercial brewer's yeast Saccharomyces cerevisiae (SafAle S-33, LeSaffre, France) and a symbiotic culture of bacteria and yeasts (SCOBY) pellicle from Kombucha tea fermentation. All strains (except for SCOBY) were grown from glycerol vial in a food-grade yeast-peptone liquid medium, and inoculated in the reference camomile brew with optical density (OD at 600 nm) of 0.5. All the inoculated infusions were stored at microaerobic condition at 30° C. for 24 h. After the incubation, each fermented infusion underwent filter sterilization (0.2 μm).

### Example 2: Sensory Analysis of Camomile Fermented with Different Yeasts

The sensory profiles of different variations of camomile infusions were tasted. The control tasted floral and green with earthy notes and a subtle sweetness. The camomile infusion fermented with Saccharomyces cerevisiae had a malty beer-like character. The camomile fermented with SCOBY had a very sharp acidity. The camomile infusions fermented with the two different Pichia yeasts had an enhanced sweetness, some banana/fruity notes and had greatly reduced green and earthy notes compared to the control. No buttery and fatty off notes were identified in the camomile fermented with Pichia yeasts. Camomile fermented with Pichia kluyveri NYSC 5485 was found to have a cleaner aroma profile to that fermented with Pichia kluyveri NCYC 246 and a less pronounced banana note.

### Example 3: Analysis of Flavor Profiles with a Focus on Aroma Compounds Present in Camomile Fermented with Different Yeasts

Quantitative volatile profiling of the camomile infusions was conducted by headspace/solid phase microextraction coupled with gas chromatogram and mass spectrometry (HS/SPME-GC-MS). The apparatus used was a gas chromatograph (Agilent Technology 8890 GC System) coupled with a mass spectrometer (Agilent Technology 7010B TQ).

Initially, 1.0 ml of the camomile infusion samples were incubated in a 10-mL screw-top headspace vial at 30° C. for 10 min, followed by extraction using a SPME fiber (PDMS/DVB, Supelco) at 30° C. for 10 min. All vials were placed in a tray cooled at 6° C. before analysis. The volatiles were thermally desorbed from the SPME fiber at 250° C. in splitless mode and resolved with a capillary column (DB-WAX, Agilent). The gas chromatograph oven temperature was programed initially at 35° C. for 5 min, then raised to 230° C. at 4° C./min, then at 230° C. for 10 min. Helium was used as the carrier gas at a flow rate of 1 mL/min. The targeted compounds were identified by pure standards and quantified in MSD ChemStation software (Agilent). All samples were measured in duplicate.

Levels of 3-methylbutyl acetate, 3-methylbutanol, 3-methylbutanal, 2-methylbutyl acetate, 2-methylbutanol, 2-methylbutanal, isobutyl acetate, 2-phenylethyl acetate, 2-phenylethanol, 2,3-butanedione, ethanol and acetic acid in parts per million after 24 hours are listed in the table below.

| | Control | P. kluyveri | P. kluyveri | P. kluyveri | S. cerevisiae | |
|---|---|---|---|---|---|---|
| Molecule in ppm | 24 H | NYSC 5485 | NCYC 246 | NCYC 2781 | S-33 | SCOBY |
| 3-methylbutyl acetate | 0.01 | 22.61 | 14.93 | 0.13 | 0.1 | 0.01 |
| 3-methylbutanol | 0 | 3.51 | 2.08 | 0.10 | 23.77 | 0.14 |
| 3-methylbutanal | 0.02 | 0.001 | 0.001 | 0.001 | 0.017 | 0.004 |
| 2-methylbutyl acetate | 0.01 | 15.45 | 10.52 | 0.05 | 0.08 | 0.01 |
| 2-methylbutanol | 0.008 | 3.978 | 2.373 | 2.243 | 13.918 | 0.075 |
| 2-methylbutanal | 0.029 | 0.002 | 0.002 | 0 | 0.013 | 0.005 |
| isobutyl acetate | 0 | 1.29 | 1.12 | 0.024 | 0.01 | 0 |
| isobutanol | 0.01 | 11.71 | 9.84 | 1.46 | 15.79 | 0.1 |
| isobutanal | 0.03 | 0.01 | 0.01 | 0.01 | 0.04 | 0.01 |
| 2-phenylethyl acetate | 0 | 4.06 | 5.42 | 0 | 0.03 | 0 |
| 2-phenylethanol | 0.02 | 2.08 | 1.12 | 2.39 | 7.03 | 0.11 |
| 2,3-butanedione | 0.079 | 0.052 | 0.094 | 0.031 | 0.153 | 0.153 |
| ethanol | 0 | 76 | 39 | 882 | 458 | 143 |
| acetic acid | 7 | 0 | 0 | 0 | 1 | 6103 |

The sample fermented with SCOBY had a high level of acetic acid, corresponding to 200 g/kg on a dry basis.

The weight ratios A, B, C and D of the aroma components are in the following table.

$Ratio A = \frac{[ 3 - meth y l butyl acetate ] + [ 3 - meth y l b u t a nol ]}{[ 3 - meth y l b u t a nal ]}$
$Ratio B = \frac{[ 2 - meth y l butyl acetate ] + [ 2 - meth y l b u t a nol ]}{[ 2 - meth y l b u t a nal ]}$
$Ratio C = \frac{[ isobutyl acetate ] + [ i s o b u t a n o l ]}{[ i s o b u t a n a l ]}$
$Ratio D = \frac{\begin{matrix}[ 3 - m e t h y l butyl acetate ] + [ 2 - m e t h y l butyl acetate ] + \\ [ isobutyl acetate ] + [ 2 - p h e n y l ethyl acetate ] + \\ [ 2 - p h e n y l e t h a n o l ]\end{matrix}}{[ 2,3 - butanedione ]}$

| | Con- | P. kluyveri | P. kluyveri | P. kluyveri | S. | |
|---|---|---|---|---|---|---|
| | trol | NYSC | NCYC | NCYC | cerevisiae | |
| Ratio | 24 H | 5485 | 246 | 2781 | S-33 | SCOBY |
| A | 1 | 24258 | 17494 | 160 | 1 397 | 44 |
| B | 1 | 9433 | 5886 | 3398 | 1057 | 19 |
| C | 0 | 1027 | 743 | 272 | 364 | 10 |
| D | 0 | 881 | 352 | 83 | 47 | 1 |

The samples fermented with a Pichia yeast (NYSC5485 and NCYC246) show higher ratios A, B, C and D than the control and the samples fermented with S. cerevisiae or SCOBY. P. kluyveri NYSC 5485 delivers a significantly higher value for ratios A, B, C and D than the type strain P. kluyveri NCYC 246 or P. kluyveri NCYC 2781.

The level of the terpene myrcene present in the samples was measured. The compound was identified through NIST library and retention index. The peak area was integrated across samples, and the ratio of fermented sample peak area against non-fermented sample peak area is shown below.

All samples were measured in duplicate.

| | P. kluyveri | P. kluyveri | S. cerevisiae | |
|---|---|---|---|---|
| | NYSC 5485 | NCYC 246 | S-33 | SCOBY |
| Myrcene | 20.5% | 139.4% | 85.7% | 106.3% |

Myrcene has a green, woody, “hop-like” note (it is also present in hops and cannabis). These notes are not always appreciated by consumers. After fermentation with Pichia kluyveri yeasts the myrcene (associated with hop-like character) was significantly reduced. P. kluyveri NYSC 5485 was found to reduce myrcene, in contrast to the type strain P. kluyveri NCYC 246 which didn't.

### Example 4: Sensory Impact of Aroma Ratio Investigated by Spiking

Solutions were made up with additions of 3-methylbutyl acetate, 3-methlybutanol and 3-methylbutanal at different levels. In one set of solutions the compounds were added to water, while in the other set, the compounds were added to the unfermented camomile extract (Ref).

The samples were tasted by a technical taste panel of 4 persons and comments compiled. The samples and comments are below.

| | 3-methylbutyl | | | | |
|---|---|---|---|---|---|
| Matrix | acetate | 3-methlybutanol | 3-methylbutanal | Ratio A | Comment |
| Water | 30 | 10 | 0.004 | 10000 | Banana, fruity, solvent-like |
| Water | 6 | 2 | 0.004 | 2000 | Low fruity and banana intensity |
| Water | 30 | 10 | 0.02 | 2000 | Higher solvent and malty notes |
| Water | 30 | 10 | 0.2 | 200 | Fruity, slight malty |
| Water | 30 | 10 | 2 | 20 | malty, less fruity |
| Camomile | 30 | 10 | 0.004 | 10000 | Clean fruity, higher mint perceived. |
| Infusion | | | | | Tea note is covered. |
| Camomile | 6 | 2 | 0.004 | 2000 | Smells like tea |
| Infusion | | | | | |
| Camomile | 30 | 10 | 0.02 | 2000 | Banana, medium tea note |
| Infusion | | | | | |
| Camomile | 30 | 10 | 0.2 | 200 | More intense tea, less fruit |
| Infusion | | | | | |
| Camomile | 30 | 10 | 2 | 20 | Higher intensity of tea and grassy |
| Infusion | | | | | notes. Lost the fruity note. |

It is important to have the right balance between ester/alcohol and the aldehyde. It was observed that in camomile infusion, high levels of ester with low levels of aldehyde, for example Ratio A greater than 2000, was able to mask the “tea” note.

### Example 5: Comparison of Genetic Sequence of P. kluyveri NYSC 5485 to the Type Strain P. kluyveri NCYC 246

The genetic sequence of Pichia kluyveri NYSC 5485 (CNCM I-5525) and P. kluyveri NCYC 246 were analysed using PacBio sequencing technology. DNA purification was performed using a Qiagen Gentra Puregene Yeast kit. Twelve DNA fragments were identified for P. kluyveri NYSC 5485 and 15 for the type strain P. kluyveri NCYC 246. The genetic sequences for P. kluyveri NYSC 5485 are filed in electronic form as SEQ ID NO:1-SEQ ID NO:12 and the genetic sequences for P. kluyveri NCYC 246 filed as SEQ ID NO:13-SEQ ID NO:27. The mitochondrion DNA was SEQ ID NO:4 for P. kluyveri NYSC 5485 and SEQ ID NO:20 for P. kluyveri NCYC 246.

The genetic sequence of Pichica kluyveri NYSC 5485 (CNCM 1-5525) was compared with that of P. kluyveri NCYC 246 using the software OrthoANlu. The global average nucleotide identity was found to be 97.91%.

Correspondences between the different sequences are given in the table below. Some sequences for Pichica kluyveri NYSC 5485 correspond most closely with a combination of sequences for P. kluyveri NCYC 246 and vice versa.

| Pichia kluyveri | P. kluyveri | Average nucleotide |
|---|---|---|
| NYSC 5485 | NCYC 246 | identity |
| SEQ ID NO: 7 | SEQ ID NO: 16 | 98.16% |
| SEQ ID NO: 10 | | |
| SEQ ID NO: 5 | SEQ ID NO: 22 | 97.90% |
| SEQ ID NO: 12 | | |
| SEQ ID NO: 11 | | |
| SEQ ID NO: 1 | SEQ ID NO: 13 | 97.87% |
| | SEQ ID NO: 15 | |
| SEQ ID NO: 3 | SEQ ID NO: 14 | 97.73% |
| SEQ ID NO: 8 | SEQ ID NO: 17 | |
| SEQ ID NO: 6 | SEQ ID NO: 27 | 97.94% |
| SEQ ID NO: 9 | | |
| SEQ ID NO: 2 | | |
| SEQ ID NO: 4 | SEQ ID NO: 20 | 99.02% |

The type strain P. kluyveri NCYC 246 comprised a genetic sequence with a maximum identity to each SEQ ID NO:1-12 taken individually as follows: SEQ ID NO:1, 97.85%; SEQ ID NO:2, 97.40%; SEQ ID NO:3, 97.86%; SEQ ID NO:4, 99.02%; SEQ ID NO:5, 98.05%; SEQ ID NO:6, 97.93%; SEQ ID NO:7, 98.12%; SEQ ID NO:8, 97.07%; SEQ ID NO:9, 97.20%; SEQ ID NO:10, 98.33%; SEQ ID NO:11, 97.526%; SEQ ID NO:12, 97.77%.

1. A beverage composition comprising fermented camomile or fermented camomile extract wherein the weight ratio of the sum of 3-methylbutyl acetate and 3-methylbutanol to 3-methylbutanal is greater than 2000.

2. The beverage composition of claim 1 wherein the weight ratio of the sum of 2-methylbutyl acetate and 2-methylbutanol to 2-methylbutanal is greater than 2000.

3. The beverage composition of claim 1 wherein the weight ratio of the sum of isobutyl acetate and isobutanol to isobutanal is greater than 400.

4. The beverage composition of claim 1 wherein the weight ratio of the sum of 3-methylbutyl acetate, 2-methylbutyl acetate, isobutyl acetate, 2-phenylethyl acetate and 2-phenylethanol to 2,3-butanedione is greater than 60.

5. The beverage composition of claim 1 having an ethanol content below 1.2 wt %.

6. The beverage composition of claim 1 wherein the beverage composition is selected from the group consisting of a ready to drink beverage, a beverage liquid concentrate, a soluble beverage powder, dried aromatic plant material and combinations of these.

7. (canceled)

8. A method of preparing a beverage composition comprising;
a. steeping camomile in water to form an aqueous camomile extract and steeped camomile;
b. fermenting the aqueous camomile extract in the presence or absence of the steeped camomile; and
wherein the fermentation is performed by yeast comprising Pichia yeast.

9. The method of claim 8 wherein the Pichia yeast is Pichia kuyveri.

10. The method of claim 8 wherein the Pichia yeast comprises a genetic sequence having a global nucleotide identity of at least 98% to the combination of SEQ IDs NO:1-12.

11. The method of claim 8 wherein the fermentation is performed in the absence of Saccharomyces cerevisiae.

12. The method of claim 8 comprising separating the aqueous camomile extract from the steeped camomile after fermentation.

13. The method of claim 8 wherein the steeped camomile, present during the fermentation of the aqueous camomile extract, is dried together with the aqueous camomile extract after fermentation.

14-15. (canceled)
